(19) United States
(12) Patent Application Publication
(10) Pub. No.: US 2024/0382312 A1
(43) Pub. Date: Nov. 21, 2024

(54) MOULDED ARTICLE AND METHOD OF MANUFACTURE

(71) Applicant: INVIBIO KNEES LIMITED, Thornton Cleveleys, Lancashire (GB)

(72) Inventors: Keith Adams, Thornton Cleveleys, Lancashire (GB); Paul Campbell, Thornton Cleveleys, Lancashire (GB); Harry Gaze, Thornton Cleveleys, Lancashire (GB)

(21) Appl. No.: 18/692,704

(22) PCT Filed: Aug. 1, 2022
(86) PCT No.: PCT/GB2022/052021

§ 371 Date: Mar. 15, 2024

(30) Foreign Application Priority Data
Sep. 17, 2021 (GB) 2113326.9

(51) Int. Cl. A61F 2/30; A61F 2/38; B24B 1/00; B24D 3/00

(52) U.S. Cl. CPC A61F 2/3094; A61F 2/3859; B24B 1/00; B24D 3/00

(57) ABSTRACT

A method of manufacturing a moulded article is disclosed. The method comprises forming a body comprising a polymer material. The method further comprises polishing a portion of the body with a polishing element to smooth at least one imperfection. The polishing element comprises a different polymer material to the polymer material of the body.

### TECHNICAL FIELD

The present invention relates to a method of manufacture of a moulded article and the moulded article thereof. Particularly, but not exclusively, the present invention relates to a method of manufacture of a moulded knee implant and the moulded knee implant. This may be a femoral knee implant component.

### BACKGROUND

Typically, polymer articles may be formed through moulding techniques, such as injection moulding or compression moulding. The mould results in a glossy surface, however surface imperfections can arise. In addition to surface imperfections, the area through which the polymer is injected into the mould, such as a gate may need to be removed. Removal of portions of the article can cause dulling of the surface, and increased roughness, which may be quantified as an Ra value. Ra is the arithmetic average of the absolute values of the profile height deviations from the mean line, recorded within the evaluation length. Simply put, Ra is the average of a set of individual measurements of the peaks and valleys of a surface.

Furthermore, moulding techniques can result in the article have split lines also known as parting lines. A split line is the border line at which draft angles change direction. That is, it is the dividing line that splits the core and cavity halves of a moulded part.

Methods for reducing imperfections on a polymer article include using abrasive media to remove an imperfection. Drag polishing is a method in which an article to be polished is pulled through a media mass, honing and polishing the article in the process. The article is then “dragged” through a work bowl filled with grinding or polishing media. Vibratory polishing comprises placing specially shaped pellets of abrasive media and articles to be polished into a vibratory tumbler. The vibrations cause the media to rub against the articles which then polish the articles.

Drag polishing and vibratory polishing have been found to be unsuitable for polishing polymer articles intended to be used for surgical purposes or polymer articles which include bearing surfaces which may rub against another surface in use. This is because during polishing abrasive particles of the abrasive media become free of the matrix in which they were bound and can become embedded in the surface of the polymer. The final article may contain impurities within the surface which has been polished. For example, for surgical implants or other medical tools, the embedded media can cause detrimental implications for biocompatibility either with toxicity to or mechanical irritation of surrounding tissue. Additionally, the embedded abrasive particles can result in excessive wear of a surface of the article or a corresponding surface of a further article which the first article is in contact with.

Non-abrasive rotating polishing wheels may also be used to polish an article. This is an advantageously simple method. However, this method is unsuitable for a polymer article because it utilizes friction to abrade the surface of the article. The friction generates heat, which softens the polymer. The softened polymer, rather than being removed is smeared over adjacent surface areas. Additionally, this method is likely to change the degree of crystallinity of the polymer at the surface. This can create excessive stress which may result in failure of the article when in use.

Flame polishing is a method of polishing a polymer article by exposing it to a flame or heat. The heat briefly melts the surface of the article and surface tensions smooth the surface. The method relies on melting the surface of the article which can create localized stress at the surface, thereby affecting wear properties. The melting will also change the crystallinity of the polymer at the surface. This method typically can only be applied consistently to a flat surface and has reduced precision for undulating surfaces.

It is an aim of certain examples of the present invention to solve, mitigate or obviate, at least partly, at least one of the problems and/or disadvantages associated with the prior art. Certain examples aim to provide at least one of the advantages described below.

In particular it is an aim of certain examples of the present invention to provide a technique for polishing a moulded article which reduces the amount of embedded particles in the surface.

Furthermore, it is an aim of certain examples of the present invention to provide a technique for polishing a moulded article which provides a smoothed transition between a machined surface and a moulded surface.

It is an aim of certain examples of the present invention to provide a technique for polishing a moulded article which reduces the Ra value of the moulded article compared to known techniques while maintaining biocompatibility.

### BRIEF SUMMARY OF THE INVENTION

Aspects of the invention provide a method of manufacturing a moulded article, a moulded article, and an implantable device as claimed in the appended claims.

According to an aspect of the invention there is provided a method of manufacturing a moulded article, wherein the method comprises:
forming a body comprising a polymer material; and
polishing a portion of the body with a polishing element to smooth at least one imperfection;
wherein the polishing element comprises a different polymer material to the polymer material of the body.

Suitably polishing the portion of the body comprises:
spinning the polishing element; and
bearing the spinning polishing element against a surface of the body such that the polishing element polishes the at least one imperfection.

Suitably the difference in Ra value of the polished portion of the body and an unpolished portion of the body is less than 0.8 micrometres.

Suitably spinning the polishing element comprises spinning the polishing element at a spindle speed of between 1000 rpm and 20,000 rpm and a feed rate of between 100 mm/min and 5000 mm/min; and wherein the Ra value of the moulded article is between 0.2 micrometres and 1.0 micrometres.

Suitably the different polymer material of the polishing element comprises a higher hardness than the polymer material of the body.

Suitably the polymer material of the body comprises polyetheretherketone, PEEK, and the different polymer material of the polishing element comprises an annealed PEEK or barium filled annealed PEEK.

Suitably the method further comprises the step of machining the body to form a machined edge, and wherein the polishing step comprises smoothing the machined edge.

Suitably machining the body comprises cutting a path with a cutter to remove a section of the body, and wherein the polishing element follows the path of the cutter.

Suitably prior to polishing the body, the method further comprises at least one of:
abrading the portion of the body with an abrasive media; or
smoothing the portion of the body with an etched glass rod.

Suitably the body is formed via injection moulding, and at least one imperfection is a moulding gate.

Suitably the moulded article is an implantable device, optionally a femoral knee component.

According to a further aspect of the invention there is provided a polishing element for use in the method of manufacturing a moulded article.

Suitably the polishing element comprises a bullnose for contacting the body.

According to a yet further aspect of the invention there is provided a moulded article manufactured by:
forming a body comprising a polymer material; and polishing a portion of the body with a polishing element to smooth at least one imperfection;
wherein the polishing element comprises a different polymer material to the polymer material of the body.

Suitably the difference in Ra value of the polished portion of the body and an unpolished portion of the body is less than 0.8 micrometres.

According to a yet further aspect of the invention there is provided an implantable device formed by:
forming a body comprising a polymer material; and
polishing a portion of the body with a polishing element to smooth at least one imperfection;
wherein the polishing element comprises a different polymer material to the polymer material of the body.

Suitably the difference in Ra value of the polished portion of the body and an unpolished portion of the body is less than 0.8 micrometres.

Suitably polishing the portion of the body comprises;
spinning the polishing element at a spindle speed of between 1000 rpm and 20,000 rpm and a feed rate of between 100 mm/min and 5000 mm/min; and
bearing the spinning polishing element against a surface of the body such that the polishing element polishes the at least one imperfection;
wherein the Ra value of the formed implantable device is between 0.2 micrometres and 1.0 micrometres.

Suitably the implantable device includes a femoral component for a knee implant.

### BRIEF DESCRIPTION OF THE DRAWINGS

Examples of the invention are further described hereinafter with reference to the accompanying drawings, in which:

FIG. 1a is a schematic illustration of a polishing element according to an example of the present invention;

FIG. 1b is a schematic illustration of a body according to an example of the present invention, formed during a process of manufacturing a moulded article;

FIG. 2 is a flow chart of a method of manufacturing a moulded article according to an example of the present invention;

FIG. 3 is a further flow chart of a method of manufacturing a moulded article according to an example of the present invention;

FIG. 4 is a yet further flow chart of a method of manufacturing a moulded article according to an example of the present invention;

FIGS. 5a to 5c are 3D illustrations of a femoral knee implant;

FIG. 6 is a schematic illustration of the femoral knee implant of FIGS. 5a to 5c; and

FIGS. 7a to 7f are Scanning Electron Microscope (SEM) images showing the surface of a femoral knee implant.

### DETAILED DESCRIPTION

Prosthetic implants can be used to partially or completely replace diseased and/or damaged joint tissue. Such implants may include articulating surfaces that replace the bone's natural articulating surfaces. For example, an implant for a knee replacement may include a femoral implant and/or a tibial implant. A femoral implant may be implanted on the distal end of the femur and may replace the articulating surfaces of the femur. A tibial implant may be implanted on the proximal end of the tibia and may replace the articulating surfaces of the tibia.

In operation, the articulating surfaces of the femoral implant articulate against the articulating surfaces of the tibial implant. Various materials have been used for the femoral and tibial implants. For example, the implants may be made from metal, for instance, cobalt-chrome. Metal implants may have a significantly higher stiffness and tensile strength than bone. As a result, metal implants can have an increased tendency to shield the underlying bone from stresses that would normally be applied to the joint during use. According to Wolff's law, bone remodels in response to applied loads. If a bone is shielded from these loads, the bone will not be exposed to the stimulus required to maintain bone mass. This can lead to a loss of bone mass that may e.g. increase the chances of the implant coming loose. In recent years, there has been increasing interest in knee implants formed from polymeric compositions that may be formulated to have mechanical properties that are more compatible with the mechanical properties of bone.

The description below describes a femoral knee implant as an example of a moulded article, however any article formed by moulding is envisaged. Example articles include, but are not limited to, implantable devices such as femoral or tibial knee implants and spinal implants, and manufacturing components such as bearings and gears.

Throughout the specification reference is made to polymer material. Preferably, the polymer is a polyaryletherketone (PAEK) material.

Suitable polyaryl ether ketone may have repeating units of formula (I) below:

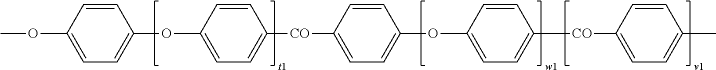

where t1 and w1 are independently represent 0 or 1 and v1 represents 0. 1 or 2.

The polyaryletherketone suitably includes at least 90, 95 or 99 mol % of repeat unit of formula I. The polyaryletherketone suitably includes at least 90, 95 or 99 weight % of repeat unit of formula I.

The polyaryletherketone may comprise or consist essentially of a repeat unit of formula I. Preferred polymeric materials comprise (or consist essentially of) a said repeat unit wherein t1=1, v1=0 and w1=0; t1=0, v1=0 and w1=0; t1=0, w1=1, v1=2; or t1=0, v1=1 and w1=0. More preferably, the polyaryletherketone comprises (e.g. consists essentially of) the repeat unit I, wherein t1=1, v1=0 and w1=0; or t1=0, v1=0 and w1=0. The most preferred polyaryletherketone comprises (especially consists essentially of) a said repeat unit wherein t1=1 v1=0 and w1=0.

The polyaryletherketone may be selected from polyetheretherketone, polyetherketone, polyetherketoneetherketoneketone and polyetherketoneketone. In some examples, the polymer is selected from polyetherketone and polyetheretherketone. The polymer is preferably polyetheretherketone (PEEK).

The polyaryletherketone may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². The Notched Izod Impact Strength, measured as mentioned above, may be less than 10 KJm⁻², suitably less than 8 KJm⁻². The Notched Izod Impact Strength, measured as mentioned above, may be at least 3 KJm⁻², suitably at least 4 KJm⁻², preferably at least 5 KJm⁻². The impact strength may be less than 50 KJm⁻², suitably less than 30 KJm⁻².

The polyaryletherketone may have a melt viscosity (MV) of at least 0.06 kNsm⁻², preferably has a MV of at least 0.09 kNsm⁻², more preferably at least 0.12 kNsm⁻², especially at least 0.15 kNsm⁻². Advantageously, the MV may be at least 0.35 kNsm⁻², and especially at least 0.40 kNsm⁻². An MV of 0.45 kNsm⁻², may be particularly advantageous.

Unless otherwise stated, melt viscosity (MV) is measured using a Bohlin Instruments RH2000 capillary rheometer according to ISO 1 1443 operating at 340° C. and a shear rate of 1000 s⁻¹ using a 0.5 mm (capillary diameter)×8.0 mm (capillary length) die with entry angle 180° C. Granules may be loaded into the barrel and left to pre-heat for 10 minutes. The viscosity may be measured once steady state conditions are reached and maintained, nominally 5minutes after the start of the test. The polyaryletherketone may have a MV of less than 1.00 kNsm⁻², preferably less than 0.5 kNsm⁻². The polyaryletherketone may have a MV in the range 0.09 to 0.5 kNsm⁻², preferably in the range 0.14 to 0.5 kNsm⁻², more preferably in the range 0.4 to 0.5 kNsm⁻².

The polyaryletherketone may have a tensile strength, measured in accordance with IS0527 (specimen type 1 b) tested at 23° C. at a rate of 50 mm/minute of at least 20 MPa, preferably at least 60 MPa, more preferably at least 80 MPa. The tensile strength is preferably in the range 80-1 10 MPa, more preferably in the range 80-100 MPa.

The polyaryletherketone may have a flexural strength, measured in accordance with IS0178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa. The polyaryletherketone may have a flexural modulus, measured in accordance with IS0178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

The polyaryletherketone may be amorphous or semi-crystalline. The polyaryletherketone is preferably crystallisable. The polyaryletherketone may be semi-crystalline. The level and extent of crystallinity in a polymer may be measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning calorimetry (DSC).

The level of crystallinity of said polyaryletherketone may be at least 1%, suitably at least 3%, preferably at least 5% and more preferably at least 10%. In especially preferred embodiments, the crystallinity may be greater than 25%. It may be less than 50% or less than 40%. The main peak of the melting endotherm (Tm) of said polyaryletherketone (if crystalline) may be at least 300° C.

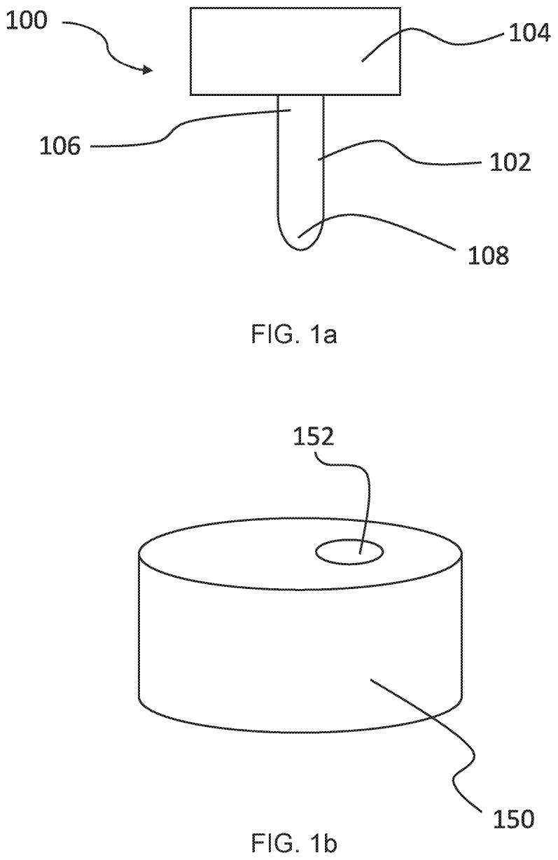

FIG. 1a illustrates a schematic example of a polishing component 100, and FIG. 1b illustrates a simple representation of a moulded body 150. In this example a surface imperfection 152 is illustrated on the moulded body 150. Once polished the body 150 comprises a manufactured moulded article. This moulded article may be an implantable device and aptly a femoral knee component.

The body 150 is formed of a polymer material. Any suitable polymer may be used to form the body 150 of the moulded article of the present disclosure. Preferably, the polymer is a polyaryletherketone (PAEK). Aptly, the polymer is polyetheretherketone (PEEK).

The polishing component 100 includes a polishing element 102 which is adapted to polish a portion of the body 150 and a driving element 104 which can spin, actuate, vibrate or otherwise move the polishing element 102.

The polishing element 102 may include an attachment end 106 which is attached to the driving element 104 and a contact end 108. The attachment end 106 may releasably connect to the driving element 104. For example, the attachment end 106 may be threaded. The polishing element 102 may therefore be replaced or retrofitted to existing driving elements. The contact end 108 of the polishing element 102 is the portion of the polishing element 102 which bears against the article body 150 in use. The driving element 104 may drive the polishing element 102 such that the contact end 108 is moving when in contact with the body 150.

In this example the contact end 108 of the polishing element 102 may be have a bullnose or otherwise be rounded. The contact 108 end may have a diameter of between 2 mm and 8 mm, and aptly between 4 mm and 5 mm. In other examples the contact end 108 may be any suitable shape or size, for example a flattened plate or a pointed cone.

In the example shown in FIG. 1a the polishing element 102 is an elongate rod. That is, the polishing element 102 has an elongate extension between the attachment end 106 and the contact end 108. It should be understood that in other examples the polishing element 102 may be a plate shape or any other suitable shape.

In an example where the moulded article is an implantable device such as a femoral knee implant, the polishing element 102 may be a rod shape and may have a bullnose contact end 108 with a diameter of between 2 mm and 8 mm, and aptly between 4 mm and 5 mm, and may be approximately 4 mm.

The polishing component 100 may also include a controller (not shown) where the controller controls the path of the contact end 108. In other words, the polishing component 100 may use an automated system to control the polishing element 102, which reduces variations between or across articles. Accordingly, the controller may include a program comprising code for controlling the path of the polishing element 102 and a machine-readable storage storing such a program. Still further, such programs may be conveyed electronically via any medium, for example a communication signal carried over a wired or wireless connection.

The polishing element 102 is a polymer material, which is a different polymer material to the polymer material of the body 150. Any suitable polymer may be used to form the polishing element 102 of the moulded article of the present disclosure. Preferably, the polymer is a polyaryletherketone (PAEK). Aptly, the polymer is polyetheretherketone (PEEK).

For example, the polymer material of the body 150 may be a first PAEK material and the different polymer material of the polishing element may be a different PAEK material. In some examples one or both of the polymer materials may include a filler such as an abrasive filler, for example, barium sulphate. That is, barium sulphate may be present in the polymer composition in an amount of up to 20 weight % of the total weight of the polymer composition. Alternatively, or in addition to including a filler, the polymer material of the polishing element 102 may be annealed. An annealed PEEK material is a different polymer material to a PEEK material which has not been annealed in the sense that the PEEK material has been hardened by annealing. In other words, the body 150 may be formed of a PEEK material which is unannealed and the polishing element 102 may be an annealed PEEK material. Therefore the polishing element 102 is harder than the body 150.

By having the polymer material of the body 150 and the polishing element 102 as different polymer materials of the PAEK family, such as PEEK in a non-annealed and annealed state respectively, the polishing element 102 allows the required Ra value to be achieved without the use of abrasive media which embed abrasive particles in the surface of the body. In this sense, the Ra value across the body of the article as a whole is lowered when compared with articles polished with alternative polishing techniques. As a result, the final article does not contain a significant amount of impurities within the surface which has been polished. This can result in increased longevity of the final article due to reduced wear, particularly on a bearing surface which rubs against another surface in use. Furthermore, for implantable devices, the biocompatibility of the article is not compromised.

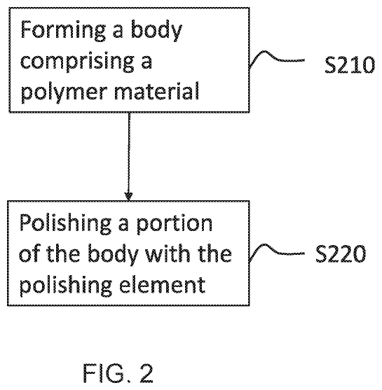

FIG. 2 shows a method of manufacturing a moulded article using the polishing component 100. The moulded article may be suitable for use as a femoral or tibial knee implant. Firstly, a body 150 comprising a polymer material is formed in step S210.

To form the body 150, a polymer material is provided which is in liquid or pliable form and shaped using a mould. Typical moulding techniques include, but are not limited to, rotational moulding, injection moulding, blow moulding, compression moulding, extrusion moulding, or thermoforming. Aptly, the moulding method may be injection moulding.

Once the body 150 is formed in step S210 at least a portion of the body may include one or more surface imperfections 152. Surface imperfections 152 may include surface bubbling, discolouration, split lines or machine edges, etc. In order to remove the surface imperfection, the portion of the body is polished with the polishing element 102 in step S220. Polishing a portion of the body 150 may include removing multiple surface imperfections 152.

Polishing the portion of the body 150 smooths the imperfection such that the difference in surface roughness of the polished area unpolished areas is smaller. That is, because the injection moulded surface is glossy (with the exception of the imperfections) the majority of the surface does not require polishing. For example, the injection moulded surface may have an average Ra value between 0.01 micrometres and 0.1 micrometres, aptly between 0.025 and 0.075 micrometres, and more aptly and average Ra value of 0.05 micrometres. The step of polishing S220 buffs the imperfection 152 so that the polished portion of the body 150 is closer to the roughness of the unpolished of the surface of the body. For example, the portion of the body which has been abraded may have a difference in Ra with an unpolished portion of the body of less than 0.8 micrometres, aptly less than 0.75 micrometres, more aptly less than 0.6 micrometres and more aptly less than 0.28 micrometres.

The Ra value of the polished portion of the body after the polishing step S220 is less than 1.5 micrometres, aptly between 0.1 micrometres and 1.2 micrometres, and more aptly between 0.2 micrometres and 1.0 micrometres.

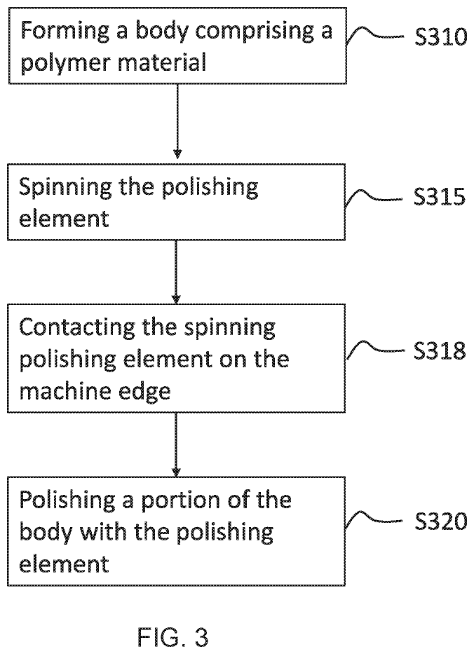

Looking to FIG. 3, a method of manufacturing the moulded article as described in FIG. 2 is illustrated, with additional steps. After forming a body comprising a polymer material in step S310, the method further comprises spinning the polishing element in step S315. Spinning the polishing element 102 may include using the driving element 104 to spin the polishing element 102. That is, the contact end 108 of the polishing element 102 may rotate about a longitudinal axis of the polishing element 102.

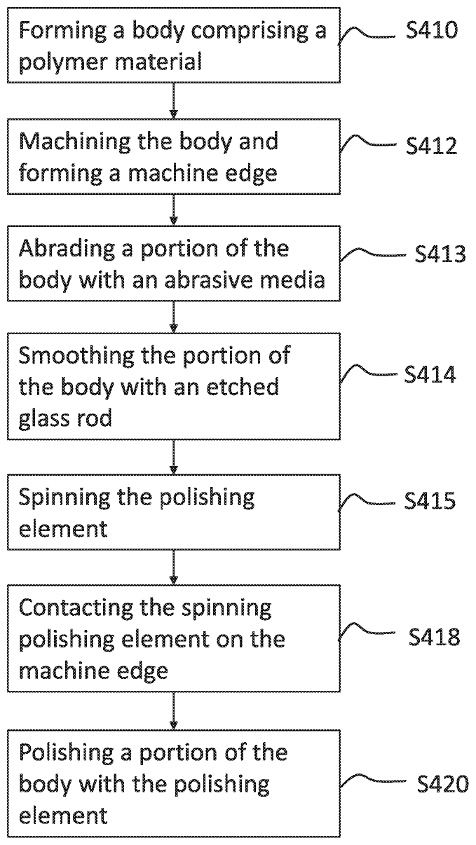

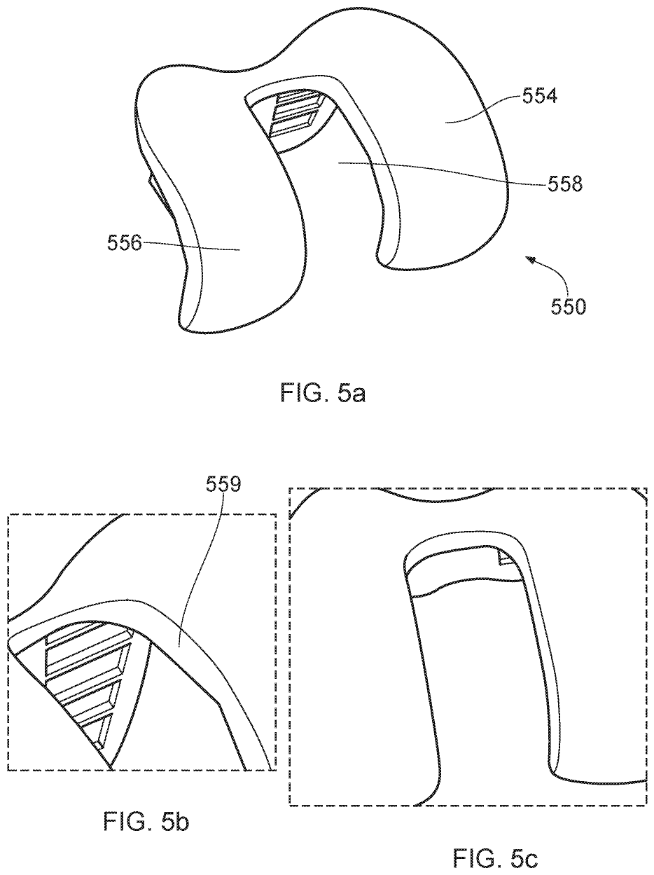

In step S315 the polishing element 102 may be spun at a spindle speed of between 1000 rpm and 20,000 rpm and a feed rate of between 100 mm/min and 5000 mm/min. Aptly the polishing element 102 may be spun at a spindle speed of between 5000 rpm and 15,000 rpm and a feed rate of between 500 mm/min and 2500 mm/min. The spin speed of the polishing element may be optimised for a specific moulded article. For example, for a femoral knee implant (shown in FIGS. 4 and 5) the spin speed may be optimised at a spindle speed of around 10,000 rpm and a feed rate of around 1000 mm/min to give a Ra value of the femoral knee implant between 0.3 micrometres and 0.5 micrometres.

The spinning polishing element 102 can then be brought to bear against a surface of the body of the moulded article in step S318 such that the polishing element 102 polishes the at least one imperfection 152. In this way, the contact end 108 of the polishing element 102 contacts the imperfection 152 and abradingly removes an area of the body 150 which includes the imperfection 152. In some examples the polishing element 102 may be set to a height below the surface of the body in order to abrade the surface imperfection 152. That is, the contact end 108 may be set to a height below that of the surface of the body, such that the contact end 108 applies a pressure to the surface of the body of the moulded article. For example the polishing element 102 may be set to a height of between 2 micrometres and 15 micrometres below the surface of the body. Aptly, the polishing element 102 may be set to a height of between 5 micrometres and 12 micrometres below the surface of the body and more aptly set at 10 micrometres below the surface of the body.

To achieve a smooth transition between the abraded area and the surface of the body 150, the abraded area may be larger than just the imperfection 152. That is, the area around the imperfection 152 may also be polished by the polishing element 102. The polishing element 102 being a different polymer material to the body 150 means that the contact end 108 abrades the imperfection 152 without embedding a significant amount of materials into the surface of the body 150 compared with abrasive polishing techniques.

Referring to FIG. 4, a method of manufacturing the moulded article as described in FIG. 3 is illustrated with additional steps. When forming a body S310 using for example an injection moulding method, the area where the polymer material enters can form a gate. Similarly, in moulding methods which include attaching two or more portions of the article together a split line may form. These imperfections may require machining away. In this example, the method includes the step of machining the body of the article S412. The step S412 of machining a body 150 may include cutting a path with a cutter to remove at least a section of the body. That is, using a cutting machine which cuts off the imperfections 152 from the body 150.

Machining the body 150 forms a machine edge, which is an area of the body 150 which is dulled or roughened by the machine. This machine edge may be an imperfection 152. The polishing element 102 may therefore polish a portion of the body 150 in step S420, which includes polishing machine edge, so as to smooth it. In some examples the polishing element 102 may follow the path of the cutter. In some examples a controller may be used to automate the machining and polishing steps S412, S420. This allows a consistency of the surface Ra value across the moulded article.

Additionally, or alternatively to the step of machining the body S412, a step S413 of abrading the body 150 with an abrasive media can be undertaken prior to polishing the body in step S420. The step S413 of abrading the body may have a further following step S414 of smoothing the portion of the body 150 with an etched glass rod. The etched glass rod may remove some embedded particles of the abrasive media from the previous step. Polishing the portion of the body 150 with the polishing element 102 may therefore be a final step S420 to give an overall smooth finish. In addition, the step S420 of polishing the portion of the body 150 may also remove embedded particles of the abrasive media from the previous steps.

In some examples micro-machining may be used for at least one of the cutting step S412 or the polishing step S420. An example of a suitable cutter is a Hurco™ five axis machine manufactured by Hurco Companies, Inc. of Pliening, Germany and described at https://www.hurco.eu/products/5-axis-machining-centers/). It will be understood that any suitable machine may be used that is capable of directing a polishing element to follow the profile of the surface to be polished.

FIGS. 5a to 5c illustrate 3D models of an example moulded article where the moulded article is a femoral knee implant.

The polymer body 550 of the femoral knee implant is moulded using injection moulding. In order to form the required shape of a femoral knee implant, the polymer is injected through a gate and split into two sections; a medial condyle 554 and a lateral condyle 556 on either side of an intercondylar slot 558. An area of the body 550 which is formed as a result of the gate is machined away removing portion of a side off the intercondylar slot 558. This portion may be between 0.1 mm and 1 mm, aptly the portion is between 0.2 mm and 0.4 mm and more aptly is 0.3 mm. This machining process also cuts into the radius creating a sharp edge 559 on the slot radius, this radius may be between 2 mm and 5 mm, aptly between 2 mm and 3 mm and more aptly 2.5 mm.

The sharp edge 559 may be polished using the methods described above, to create a smooth transition along the intercondylar slot 558.

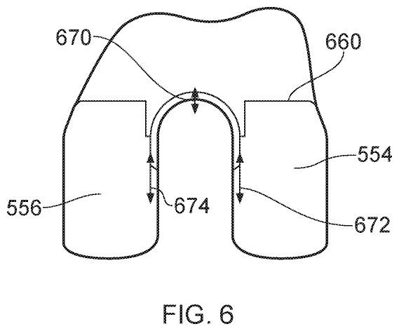

FIG. 6 illustrates a schematic image of the example moulded article of FIGS. 5a to 5c. The intercondylar slot 558 of the implant incudes a split line 660 which requires machining away using the above described polishing method.

The Ra value may be measured in three places, for example at the crux 670 of the intercondylar slot 558, and on the edge 672, 674 of the intercondylar slot 558 on both a medial condyle 554 and a lateral condyle 556. This can therefore determine if the polished portion is sufficiently smooth for use in surgical applications. For implantable devices, the above described method provides a smoothed article, which has reduced bio-incompatibility compared to methods which use abrasive media.

The method of manufacturing a moulded article as described above advantageously results in a moulded article which has reduced wear over its lifetime due to smoother surfaces. Additionally, the moulded article may have a significantly improved its appearance in the sense areas with removed imperfections match the lustre of the moulded surface.

### Example 1

Samples of a femoral knee implant were formed from a PEEK material. The samples were machined using a Hurco™ five-axis milling machine and then polished using a barium sulphate filled and annealed PEEK rod. The Ra value was measured on a plurality of femoral knee implants of the same size and variant on the medial and lateral condyles. The results are illustrated in table 1 below.

TABLE 1

| | |
|---|---|
| Machined only | Polished with PEEK rod |

| Medial | Lateral | Medial | Lateral |
|---|---|---|---|
| condyle Ra | condyle Ra | condyle Ra | condyle Ra |
| 0.537 | 0.637 | 0.436 | 0.346 |
| 0.711 | 0.735 | 0.418 | 0.306 |
| 0.905 | 0.576 | 0.179 | 0.415 |
| 0.442 | 0.564 | 0.406 | 0.258 |
| 0.913 | 0.703 | 0.381 | 0.572 |
| 0.716 | 0.445 | 0.469 | 0.343 |

The average Ra value for an implant with no polishing was 0.657 micrometres. The average Ra value after polishing with the Barium filled PEEK rod was 0.377 micrometres. This shows a significant reduction in the Ra value after polishing, resulting in an improved surface finish.

### Example 2

Samples of a femoral knee implant were formed from a PEEK material. The samples were machined using a Hurco™ five-axis milling machine and then polished using an annealed Barium filled PEEK rod or an annealed PEEK only rod. The Ra value was measured on the medial and lateral condyles. The average Ra value of the samples polished with the annealed Barium filled PEEK rod was 0.751 micrometres and the average Ra value the samples polished with the annealed PEEK rod was 0.7509 micrometres. This shows no significant difference between samples polished with an annealed PEEK only rod or an annealed Barium filled PEEK rod.

### Example 3

Samples of a femoral knee implant were formed from a PEEK material. The samples were machined using a Hurco™ five-axis milling machine and then polished using an annealed Barium filled PEEK rod or an annealed PEEK only rod. The spindle speed and feed rate were optimised to a spindle speed of 10,000 rpm and a feed rate of 1000 mm/min.

The Ra value on the machined and polished areas of the medial and lateral condyles was measured to see how rough the machine and polish process with each rod type made the surface. The average Ra value of the samples polished with the Barium filled PEEK rod was 0.36 micrometres and the average Ra value the samples polished with the PEEK rod was 0.39 micrometres. This shows no significant difference between samples polished with an annealed PEEK only rod or an annealed Barium filled PEEK rod.

### Example 4

Samples of a femoral knee implant were formed from a PEEK material. Some of the samples were machined using a Hurco™ five-axis milling machine and then polished using an annealed Barium filled PEEK rod. Other samples were machined using a Hurco™ five-axis milling machine and then polished by hand using abrasive papers of P800 paper followed by P2500 and then a felt pad.

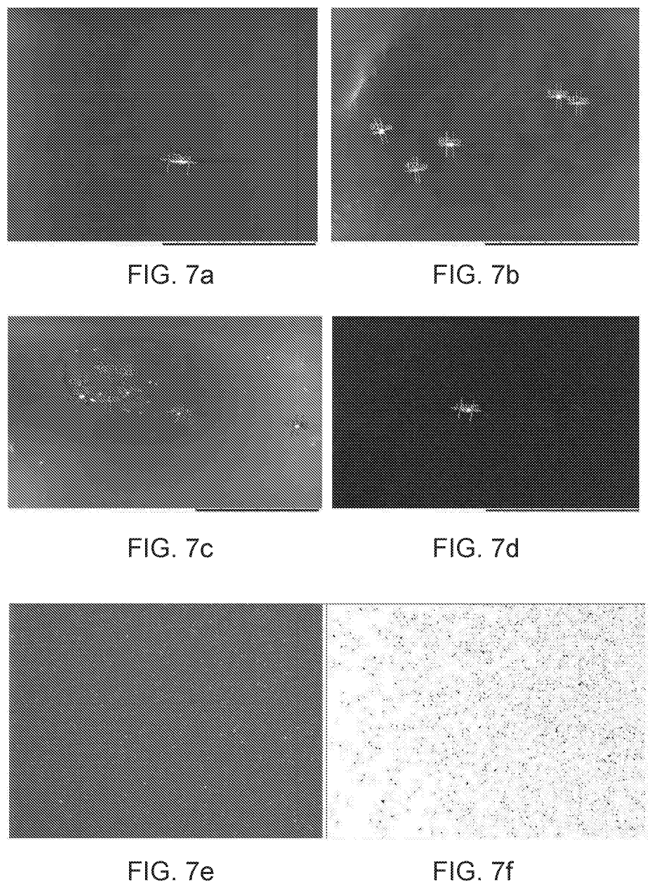

The machined and polished samples were analysed using a Scanning Electron Microscope (SEM). The results are illustrated in FIGS. 7a to 7f. FIGS. 7a to 7d illustrate the surface of the sample which was polished using the Barium filled PEEK rod. FIG. 7e illustrates the surface polished by hand and FIG. 7f is FIG. 7e with particle isolation.

For the samples polished with the annealed Barium filled PEEK rod A small number of barium sulphate particles were found on the surface. The largest particle found was 2.63 micrometres, with the majority sub-micron. In contrast, for the samples hand polished a significant number of aluminium oxide, iron and sodium particles were found. The largest particle was measured to be 40.829 micrometres.

Therefore, although a small amount of debris was introduced by the annealed Barium filed PEEK rod, the amount and particle size were extremely small when compared with alternative polishing methods.

Throughout this specification, the words “comprise” and “contain” and variations of them mean “including but not limited to”, and they are not intended to (and do not) exclude other components, integers or steps. Throughout this specification, the singular encompasses the plural unless the context otherwise requires. In particular, where the indefinite article is used, the specification is to be understood as contemplating plurality as well as singularity, unless the context requires otherwise. Throughout this specification, the term “about” is used to provide flexibility to a range endpoint by providing that a given value may be “a little above” or “a little below” the endpoint. The degree of flexibility of this term can be dictated by the particular variable and can be determined based on experience and the associated description herein.

Concentrations, dimensions, amounts, and other numerical data may be presented herein in a range format. It is to be understood that such range format is used merely for convenience and brevity and should be interpreted flexibly to include the numerical values explicitly recited as the limits of the range, and also to include all the individual numerical values or sub-ranges encompassed within that range as if the numerical value and sub-range is recited. For example, a weight ratio range of about 1 wt % to about 20 wt % should be interpreted to include the explicitly recited limits of 1 wt % and about 20 wt %, and also to include individual weights such as 2 wt %, 11 wt %, 14 wt %, and sub-ranges such as 10 wt % to 20 wt %, 5 wt % to 15 wt %, etc.

Features, integers or characteristics described in conjunction with a particular aspect or example of the invention are to be understood to be applicable to any other aspect or example described herein unless incompatible therewith. All of the features disclosed in this specification, and/or all of the steps of any method or process so disclosed, may be combined in any combination, except combinations where at least some of such features and/or steps are mutually exclusive. The invention is not restricted to the details of any foregoing examples. The invention extends to any novel feature or combination of features disclosed in this specification. It will be also be appreciated that, throughout this specification, language in the general form of “X for Y” (where Y is some action, activity or step and X is some means for carrying out that action, activity or step) encompasses means X adapted or arranged specifically, but not exclusively, to do Y.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The reader's attention is directed to all papers and documents which are filed concurrently with or previous to this specification in connection with this application and which are open to public inspection with this specification, and the contents of all such papers and documents are incorporated herein by reference.

1. A method of manufacturing a moulded article, wherein the method comprises:
forming a body comprising a polymer material; and
polishing a portion of the body with a polishing element to smooth at least one imperfection;
wherein the polishing element comprises a different polymer material to the polymer material of the body.

2. The method as claimed in claim 1, wherein polishing the portion of the body comprises:
spinning the polishing element; and
bearing the spinning polishing element against a surface of the body such that the polishing element polishes the at least one imperfection.

3. The method as claimed in claim 1, wherein the difference in Ra value of the polished portion of the body and an unpolished portion of the body is less than 0.8 micrometres.

4. The method as claimed in claim 2, wherein spinning the polishing element comprises spinning the polishing element at a spindle speed of between 1000 rpm and 20,000 rpm and a feed rate of between 100 mm/min and 5000 mm/min; and
wherein the Ra value of the polished portion of the body is between 0.2 micrometres and

1. 0 micrometres.

5. The method as claimed in any preceding claim 1, wherein the different polymer material of the polishing element comprises a higher hardness than the polymer material of the body.

6. The method as claimed in claim 5, wherein the polymer material of the body comprises polyetheretherketone, PEEK, and the different polymer material of the polishing element comprises an annealed PEEK or Barium filled annealed PEEK.

7. The method as claimed in claim 1, wherein the method further comprises the step of machining the body to form a machined edge, and wherein the polishing step comprises smoothing the machined edge.

8. The method as claimed in claim 7, wherein machining the body comprises cutting a path with a cutter to remove a section of the body, and wherein the polishing element follows the path of the cutter.

9. The method as claimed in claim 1, wherein prior to polishing the body, the method further comprises at least one of:
abrading the portion of the body with an abrasive media; or
smoothing the portion of the body with an etched glass rod.

10. The method as claimed in claim 1, wherein the body is formed via injection moulding, and at least one imperfection is a moulding gate.

11. The method as claimed in claim 1, wherein the moulded article is an implantable device.

12. A polishing element for use in the method of manufacturing a moulded article as claimed in claim 1.

13. A polishing element as claimed claim 12, wherein the polishing element comprises a bullnose for contacting the body.

14. A moulded article manufactured by:
forming a body comprising a polymer material; and
polishing a portion of the body with a polishing element to smooth at least one imperfection;
wherein the polishing element comprises a different polymer material to the polymer material of the body.

15. The moulded article of claim 14, wherein the difference in Ra value of the polished portion of the body and an unpolished portion of the body is less than 0.8 micrometres.

16. An implantable device formed by:
forming a body comprising a polymer material; and
polishing a portion of the body with a polishing element to smooth at least one imperfection;
wherein the polishing element comprises a different polymer material to the polymer material of the body.

17. The implantable device of claim 16, wherein the difference in Ra value of the polished portion of the body and an unpolished portion of the body is less than 0.8 micrometres.

18. The implantable device as claimed in claim 17, wherein polishing the portion of the body comprises:
spinning the polishing element at a spindle speed of between 1000 rpm and 20,000 rpm and a feed rate of between 100 mm/min and 5000 mm/min; and
bearing the spinning polishing element against a surface of the body such that the polishing element polishes the at least one imperfection;
wherein the Ra value of the polished portion of the body is between 0.2 micrometres and 1.0 micrometres.

19. The implantable device of claim 16, wherein the implantable device includes a femoral component for a knee implant.

20. The method as claimed in claim 11, wherein the implantable device is a femoral knee component.
